SEQUENCE LISTING (2 sequences)

SEQ ID NO: 1 - protein, 140 aa, synthetic construct
  REGION 1..140: note = Synthetic
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 146 aa, synthetic construct
  REGION 1..146: note = Synthetic
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEAHHHHHH

(19) United States
(12) Patent Application Publication
(10) Pub. No.: US 2024/0085435 A1
(43) Pub. Date: Mar. 14, 2024

(54) ALPHA-SYNUCLEIN SEED AMPLIFICATION ASSAY FOR PERIPHERAL MATRICES

(71) Applicant: Amprion, Inc., San Francisco, CA (US)

(72) Inventors: Luis Concha, San Diego, CA (US); Carly Farris, San Diego, CA (US); Yihua Ma, Corona, CA (US)

(73) Assignee: Amprion, Inc., San Francisco, CA (US)

(21) Appl. No.: 18/464,367

(22) Filed: Sep. 11, 2023

Related U.S. Application Data
(63) Continuation-in-part of application No. 17/346,854, filed on Jun. 14, 2021.
Application No. 17/346,854 is a continuation of application No. 17/011,374, filed on Sep. 3, 2020, now Pat. No. 11,079,396.
(60) Provisional application No. 63/375,126, filed on Sep. 9, 2022.
(60) Provisional application No. 62/895,535, filed on Sep. 4, 2019.
(60) Provisional application No. 63/040,144, filed on Jun. 17, 2020.
(60) Provisional application No. 63/042,679, filed on Jun. 23, 2020.
(60) Provisional application No. 63/045,593, filed on Jun. 29, 2020.
(60) Provisional application No. 63/073,420, filed on Sep. 1, 2020.
(60) Provisional application No. 63/073,424, filed on Sep. 1, 2020.

(51) Int. Cl. G01N 33/68

(52) U.S. Cl. CPC G01N 33/6896; G01N 2800/2814; G01N 2800/2835

(57) ABSTRACT

Methods and kits are provided for reproducible detection of misfolded aS aggregates in biological fluids and tissue that are less-invasively or non-invasively obtained compared to cerebrospinal fluid, such as skin, olfactory mucosa, saliva, and blood or blood parts.

### CROSS-REFERENCE TO RELATED APPLICATIONS

This application claims the benefit of U.S. Provisional Application No. 63/375,126, filed on Sep. 9, 2022. This application is also a continuation in part of U.S. application Ser. No. 17/346,854, filed Jun. 14, 2021, which is a continuation of U.S. application Ser. No. 17/011,374, filed on Sep. 3, 2020, now issued as U.S. Pat. No. 11,079,396, which claims the benefit of U.S. Provisional Application No. 62/895,535, filed on Sep. 4, 2019; U.S. Provisional Application No. 63/040,144, filed on Jun. 17, 2020; U.S. Provisional Application No. 63/042,679, filed on Jun. 23, 2020; U.S. Provisional Application No. 63/045,593, filed on Jun. 29, 2020; U.S. Provisional Application No. 63/073,420, filed on Sep. 1, 2020; and U.S. Provisional Application No. 63/073,424, filed on Sep. 1, 2020. Each of these applications is incorporated by reference herein in its entirety.

### SEQUENCE LISTING

A Sequence Listing has been submitted electronically in XML format and is hereby incorporated by reference in its entirety. The XML copy, created on Nov. 20, 2023, is named Amprion-AS-BIOS-US-ST26.xml and is 3,822 bytes in size.

### BACKGROUND

The accumulation and deposition of α-synuclein (αS) aggregates in brain tissue is the main event in the pathogenesis of different neurodegenerative disorders referred to as synucleinopathies. Synucleinopathies include Parkinson's disease (PD), Dementia with Lewy Bodies (DLB), Multiple System Atrophy (MSA), isolated rapid eye movement (REM) sleep behavior disorder (iRBD), and Pure Autonomic Failure (PAF). Current diagnosis of these disorders mainly relies on the recognition of clinical symptoms, which unfortunately typically provide a diagnosis only when the neurodegeneration is already in an advanced phase.

Seed amplification assays (SAAs) provide an ultra-sensitive method for detecting misfolded protein aggregates through artificial acceleration and amplification of the misfolding and aggregation process in vitro. αS-SAAs have been used successfully to detect misfolded αS aggregates in cerebrospinal fluid (CSF) with very high sensitivity and specificity. See U.S. Pat. Nos. 10,989,718 and 11,079,396, each of which is incorporated by reference herein in its entirety. Recent development of methods for preparing reliable monomeric αS substrate for use in the αS-SAAs has played an important role in making αS-SAA of CSF clinically and commercially viable. See U.S. Pat. No. 11,254,718, which is incorporated by reference herein in its entirety.

While αS-SAA of CSF is the gold standard for detecting misfolded αS aggregates, obtaining CSF requires an invasive lumbar puncture, also known as a spinal tap, to remove a sample of CSF from the subarachnoid space in the spine. During a lumbar puncture, a hollow needle is inserted through the skin in the lower back. The needle passes between the vertebrae and into the spinal canal. While a lumbar puncture is safe for most people, some people get a severe headache known as a “spinal headache” caused by CSF leakage. Rare complications include back or leg pain, accidental puncture of the spinal cord, bleeding in the spinal canal, and brain herniation caused by a sudden decrease of CSF pressure.

Methods and kits are needed for reproducible detection of misfolded αS aggregates in biological fluids and tissue that are less-invasively or non-invasively obtained, such as skin, olfactory mucosa (OM), blood, tears, urine, and saliva.

### SUMMARY

In one aspect, an in vitro method for detecting the presence of αS aggregates in a skin sample is provided, the method comprising: (A) homogenizing the skin sample to form a skin homogenate; (B) providing a pre-incubation mixture, the pre-incubation mixture comprising: (1) a monomeric αS substrate; (2) a buffer composition; (3) a salt solution; (4) an indicator comprising a fluorophore; (5) sarkosyl; and (6) a bead; and (C) combining the skin homogenate and the pre-incubation mixture to form a reaction mixture; (D) incubating the reaction mixture with at least one agitation cycle to form an incubated reaction mixture; (E) illuminating the incubated reaction mixture with a wavelength of light that excites the fluorophore; and (F) determining a level of fluorescence during incubation, wherein an increase in the level of fluorescence indicates the presence of αS aggregate in the skin sample, and wherein the bead(s) is not a superparamagnetic, monosized polymer bead coated with a thin, inert polymer shell to encase the magnetic material (e.g., Dynabeads® magnetic beads).

In another aspect, an in vitro method for detecting the presence of αS aggregate in an OM sample is provided, the method comprising: (A) centrifuging or otherwise sedimenting the OM sample to form a sedimented OM sample; (B) providing a pre-incubation mixture, the pre-incubation mixture comprising: (1) a monomeric αS substrate; (2) a buffer composition; (3) a salt solution; (4) an indicator comprising a fluorophore; (5) sarkosyl; and (6) a bead; and (C) combining the sedimented OM sample and the pre-incubation mixture to form a reaction mixture; (D) incubating the reaction mixture with at least one agitation cycle to form an incubated reaction mixture; (E) illuminating the incubated reaction mixture with a wavelength of light that excites the fluorophore; and (F) determining a level of fluorescence during incubation, wherein an increase in the level of fluorescence indicates the presence of αS aggregate in the OM sample, and wherein the bead(s) is not a superparamagnetic, monosized polymer bead coated with a thin, inert polymer shell to encase the magnetic material (e.g., Dynabeads® magnetic beads). In some aspects, the OM sample is washed and/or diluted as a part of preprocessing the OM sample.

In another aspect, an in vitro method for detecting the presence of αS aggregate in a blood or blood-derived (e.g., plasma and/or serum) sample is provided, the method comprising: (A) combining the blood or blood-derived sample with αS seeds to form an immunoprecipitation mixture; (B) incubating the immunoprecipitation mixture with a population of anti-αS antibody coated beads to form incubated beads; (C) providing a pre-reaction mixture, the pre-reaction mixture comprising: (1) a monomeric αS substrate; (2) a buffer composition; (3) a salt solution; (4) an indicator comprising a fluorophore; and (5) a detergent; (D) combining the incubated beads and the pre-reaction mixture to form a reaction mixture; (E) incubating the reaction mixture with at least one agitation cycle to form an incubated reaction mixture; (F) illuminating the incubated reaction mixture with a wavelength of light that excites the fluorophore; and (G) determining a level of fluorescence during incubation, wherein an increase in the level of fluorescence indicates the presence of αS aggregate in the blood or blood-derived sample. In one aspect, the incubated beads comprise αS seeds, and the αS seeds are eluted, such that step (D) comprises combining the eluted αS seeds and the pre-reaction mixture to form the reaction mixture.

In another aspect, an in vitro method for detecting the presence of αS aggregate in a blood or blood-derived sample is provided, the method comprising: (A) centrifuging the human blood or blood-derived sample to form a pellet; (B) providing a pre-incubation mixture, the pre-incubation mixture comprising: (1) a monomeric αS substrate; (2) a buffer composition; (3) a salt solution; (4) an indicator comprising a fluorophore; and (5) sarkosyl; and (C) combining the pellet and the pre-incubation mixture to form a reaction mixture; (D) incubating the reaction mixture with at least one agitation cycle to form an incubated reaction mixture; (E) illuminating the incubated reaction mixture with a wavelength of light that excites the fluorophore; and (F) determining a level of fluorescence during incubation, wherein an increase in the level of fluorescence indicates the presence of αS aggregate in the blood or blood-derived sample. In one aspect, the pellet is re-suspended in a second buffer composition prior to combining the pellet with the pre-incubation mixture.

In another aspect, an in vitro method for detecting the presence of αS aggregate in a saliva sample is provided, the method comprising: (A) providing a saliva sample; (B) providing a pre-incubation mixture, the pre-incubation mixture comprising: (1) a monomeric αS substrate; (2) a buffer composition; (3) a salt solution; (4) an indicator comprising a fluorophore; and (5) sarkosyl; and (C) combining the saliva sample and the pre-incubation mixture to form a reaction mixture; (D) incubating the reaction mixture with at least one agitation cycle to form an incubated reaction mixture; (E) illuminating the incubated reaction mixture with a wavelength of light that excites the fluorophore; and (F) determining a level of fluorescence during incubation, wherein an increase in the level of fluorescence indicates the presence of αS aggregate in the saliva sample. In one aspect, the pre-incubation mixture further comprises a bead, with the caveat that the bead is not a superparamagnetic, monosized polymer bead coated with a thin, inert polymer shell to encase the magnetic material (e.g., Dynabeads® magnetic beads).

### BRIEF DESCRIPTION OF THE FIGURES

The claimed invention may be more readily understood by reference to the following figures, wherein:

FIG. 1 is an example depiction of an αS-SAA process applied to a biological sample, such as skin homogenate, OM, saliva, or blood or blood parts, that contains misfolded αS aggregates.

FIG. 2 is a schematic representation of an immunoprecipitation method for detecting the presence of αS aggregate in a blood plasma sample.

FIG. 3 shows αS-SAA aggregation curves using the monomeric αS substrate corresponding to SEQ ID NO. 2 in the presence of immunoprecipitated human blood plasma spiked with various concentrations of αS synthetic seeds.

FIG. 4 is a plot of time to reach 50% aggregation (T50) as a function of the logarithm of αS synthetic seed mass for the immunoprecipitation and aggregation study referred to in FIG. 3.

FIG. 5 shows the maximum ThT fluorescence (left) during αS-SAA using the monomeric αS substrate corresponding to SEQ ID NO: 2 in the presence of immunoprecipitated human blood plasma from patients diagnosed as having PD compared to healthy controls (HCs). Statistical significance was evaluated by t-test (right).

FIG. 6 shows the aggregation curves resulting from centrifugation of human blood plasma spiked with various concentrations of αS synthetic seeds, resuspension of the pellets in a pre-incubation mixture, and αS-SAA of the resultant reaction mixtures.

FIG. 7 is a plot of time to reach T50 as a function of the logarithm of αS synthetic seed mass for the sedimentation/aggregation study referred to in FIG. 6.

FIG. 8 shows αS-SAA aggregation curves using the monomeric αS substrate corresponding to SEQ ID NO. 2 in the presence of a sedimented human blood plasma sample from a patient diagnosed as having PD compared to a HC.

FIG. 9 is a graph showing maximum ThT fluorescence during αS-SAA using the monomeric αS substrate corresponding to SEQ ID NO. 2 in the presence of sedimented human blood plasma samples from patients diagnosed as having PD compared to HCs.

FIG. 10 is a graph showing maximum ThT fluorescence during αS-SAA using the monomeric αS substrate corresponding to SEQ ID NO. 2 in the presence of sedimented human blood plasma samples from patients diagnosed as having PD compared to HCs.

FIG. 11 shows αS-SAA aggregation curves using the monomeric αS substrate corresponding to SEQ ID NO. 2 in the presence of skin homogenates spiked with various concentrations of synthetic αS seeds.

FIG. 12 shows αS-SAA aggregation curves using the monomeric αS substrate corresponding to SEQ ID NO. 2 in the presence of a skin homogenates from patients diagnosed as having various synucleinopathies compared to non-synucleinopathy controls.

FIGS. 13A-13C show αS-SAA aggregation curves under different centrifugation conditions from diluted skin homogenate from a patient diagnosed as having PD. FIGS. 13D and 13E show the αS-SAA aggregation curves from the skin and CSF, respectively, of the same patient diagnosed as having PD. FIGS. 13F and 13G show αS-SAA aggregation curves from skin homogenates from patients diagnosed as having PD and MSA, respectively. FIG. 13H shows S-SAA aggregation curves from skin homogenates from a non-synucleinopathy (NS) control subject.

FIGS. 14A-B show αS-SAA aggregation curves using the monomeric αS substrate corresponding to SEQ ID NO. 2 in the presence of an OM sample from a patient diagnosed as having PD (FIG. 14A) compared to a non-synucleinopathy control (FIG. 14B).

FIGS. 15A-15B show αS-SAA aggregation curves using the monomeric αS substrate corresponding to SEQ ID NO. 2 in the presence of a HC saliva sample spiked with CSF from a patient diagnosed as having PD (FIG. 15A) compared to a HC saliva sample spiked with HC CSF (FIG. 15B).

### DETAILED DESCRIPTION

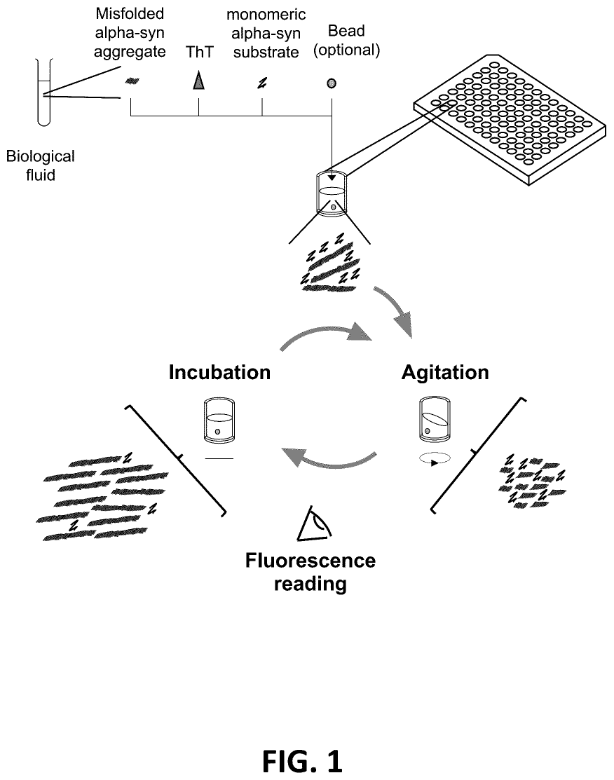

αS-SAA methods and kits are provided for determining the presence of misfolded αS aggregates in a biological sample, in vitro. Generally, the αS-SAA methods and kits comprise providing a pre-incubation mixture, the pre-incubation mixture comprising: a monomeric αS substrate; a buffer composition; a salt solution; and an indicator comprising a fluorophore. The αS-SAA methods and kits further comprise combining the biological sample and the pre-incubation mixture to form a reaction mixture; incubating the reaction mixture with at least one agitation cycle to form an incubated reaction mixture; illuminating the incubated reaction mixture with a wavelength of light that excites the fluorophore; and determining a level of fluorescence during incubation, wherein an increase in the level of fluorescence indicates the presence of αS aggregate in the biological sample. In some aspects, the pre-incubation mixture further comprises a bead, which may be chemically inert. See, e.g., FIG. 1.

### Definitions

The term “about” in conjunction with a number is intended to include ±10% of the number. This is true whether “about” is modifying a stand-alone number or modifying a number at either or both ends of a range of numbers. In other words, “about 10” means from 9 to 11. Likewise, “about 10 to about 20” contemplates 9 to 22 and 11 to 18. In the absence of the term “about” or a clear indication of a range (e.g., ±10%) the exact number is intended. In other words, “10” means 10.

The singular forms “a,” “an,” and “the” include plural referents unless the context clearly dictates otherwise. Thus, for example, reference to “a bead” also includes a plurality of beads.

Where a range of values is provided, each intervening value, to the tenth of the unit of the lower limit unless the context clearly dictates otherwise, between the upper and lower limit of that range, and any other stated or intervening value in that stated range, is encompassed. The upper and lower limits of these smaller ranges may independently be included in the smaller ranges and are also encompassed, subject to any specifically excluded limit in the stated range. Where the stated range includes one or both of the limits, ranges excluding either or both of those included limits are also included.

“Misfolded αS aggregate” or “αS aggregate” refers to aggregates of misfolded αS protein. Aggregates may be referred to as oligomers or polymers, and aggregation may be referred to as oligomerization or polymerization.

A “misfolded αS protein” is an αS protein that lacks all or part of the structural conformation of the protein as it exists in its typical, nonpathogenic normal function within a biological system. A misfolded αS protein may aggregate. A misfolded αS protein may localize in a protein aggregate. A misfolded αS protein may be a non-functional protein. A misfolded αS protein may be a pathogenic conformer of the protein.

As used herein, “soluble” species, including soluble misfolded αS aggregate, may form a solution in biological fluids under physiological conditions, whereas “insoluble” species may be present as precipitates, fibrils, deposits, tangles, or other non-dissolved forms in such biological fluids. A species that dissolves in a non-biological fluid but not a biological fluid under physiological conditions is considered insoluble. For example, fibrils of αS and the like may be dissolved in a solution of, e.g., a surfactant such as sodium dodecyl sulfate (SDS) in water but may still be insoluble in biological fluids under physiological conditions and are, therefore, considered insoluble.

Nucleation-dependent aggregation may be characterized by a slow “lag phase,” wherein aggregated nuclei form and stimulate the rapid formation of further and/or larger aggregates. The lag phase may be minimized or eliminated by addition of pre-formed “nuclei” or “seeds.” “Seeds” or “nuclei” refer to misfolded αS protein or short fragmented fibrils with the ability to induce further aggregation.

Aggregates of misfolded αS protein may be “de-aggregated,” i.e., broken up or disrupted, to release smaller fragments and aggregates, e.g., fragmented fibrils and smaller misfolded αS aggregates. The catalytic activity of a collection of misfolded αS aggregate seeds may scale, at least in part with the number of seeds in a mixture. Accordingly, disruption of misfolded αS aggregates to release smaller misfolded αS aggregates and fragmented fibrils as seeds may lead to an increase in catalytic activity for further aggregation.

The phrases “monomeric αS protein” and “monomeric αS substrate” are used interchangeably and refer to one or more seed-free, αS protein molecules in their native, nonpathogenic configuration without the catalytic activity for aggregation associated with seeds.

Unless defined otherwise, all technical and scientific terms have the same meaning as commonly understood by one of ordinary skill in the art to which this invention belongs.

### Biological Fluids

A “biological fluid” is meant to include fluid or fluid expressed from blood or blood parts such as plasma and/or serum, skin homogenate, mucosal membrane, e.g., nasal mucosal membrane, including OM, and saliva. When a biological fluid has been removed from the body and, as applicable, processed and/or prepared for use in the methods and kits described herein, it is referred to as a “biological sample,” or simply, a “sample.” When a sample is referred to in the claims as being “provided,” e.g., a skin sample, an OM sample, or a saliva sample, the meaning that is intended is that the sample is provided in a processed and/or prepared form ready for αS-SAA, unless the context clearly indicates otherwise. The methods and kits described herein are conducted and used in vitro.

### Extracting the Misfolded αS Aggregates

In several aspects, the method may include selectively concentrating and/or extracting the misfolded αS aggregate. In some aspects, the method comprises the step of concentrating and/or extracting the misfolded αS aggregate in the biological sample before incubating the biological sample using antibodies that specifically bind to misfolded αS aggregate. Selectively concentrating and/or extracting the misfolded αS aggregate may include pre-treating the biological sample prior to forming the incubation mixture. The step of selectively concentrating and/or extracting the misfolded αS aggregate may include pre-treating the incubation mixture prior to incubating the incubation mixture. The step of selectively concentrating and/or extracting the misfolded αS aggregate may include contacting the incubation mixture with antibodies that specifically bind to soluble, misfolded αS protein to form a “captured” misfolded αS aggregate.

Antibodies are designed for specific binding. An antibody “specifically binds” when the antibody avidly binds a target structure, or subunit thereof, but binds to a substantially lesser degree or does not bind to a structure that is not the target structure. In some aspects, the antibody specifically binds to soluble, misfolded αS protein with a specific affinity of between 10⁻⁸ M and 10⁻¹¹ M. In some aspects, an antibody or antibody fragment binds to the soluble, misfolded αS protein with a specific affinity of greater than 10⁻⁷ M, 10⁻⁸ M, 10⁻⁹ M, 10⁻¹⁰ M, or 10⁻¹¹ M, between 10⁻⁸ M-10⁻¹¹ M, 10⁻⁹ M-10⁻¹⁹ M, and 10⁻¹⁹ M-10⁻¹¹ M. In one aspect, specific activity is measured using a competitive binding assay as set forth in Ausubel F M, (1994). Current Protocols in Molecular Biology. Chichester: John Wiley and Sons, which is incorporated herein by reference.

Antibodies that specifically bind to soluble, misfolded αS protein may include one or more of: α/β-syn N-19; α-syn C-20-R; α-syn 211; α-syn Syn 204; α-syn 2B2D1; α-syn LB 509; α-syn SPM451; α-syn 3G282; α-syn 3H2897; α/β-syn Syn 202; α/β-syn 3B6; α/β/γ-syn FL-140; and the like. The αS-specific antibodies may include: an antibody specific for an amino acid sequence of αS and an antibody specific for a conformation of the soluble, misfolded αS protein. The αS-specific antibodies may be coupled to a solid phase.

The solid phase may include a paramagnetic particle (e.g., iron oxide) and/or a multi-well plate. For example, ELISA plates may be coated with the antibodies used to capture αS from the biological sample. The antibody-coated ELISA plates may be incubated with a biological sample, unbound materials may be washed off, and the αS-SAA reaction may be performed on the concentrated biological sample. Antibodies may also be coupled to particles (e.g., Dynabeads). The particles may be incubated with the biological sample and used to separate αS-antibody complexes from the remainder of the biological sample.

In several aspects, the method of concentrating and/or extracting misfolded αS aggregate in biological samples may include ultracentrifugation. Misfolded αS aggregate can be pelleted from biological samples using high-speed centrifugation. The resulting pellet is re-suspended in a buffer and analyzed using the methods and kits claimed herein. The centrifugation step may include the use of detergents, such as, for example, sarkosyl, polysorbate 20, Triton, and the like.

### Monomeric αS Substrate

As used herein, “αS” may refer to full-length, 140 amino acid alpha-synuclein protein, e.g., “αS-140.” Other isoforms or fragments may include “αS-126,” alpha-synuclein-126, which lacks residues 41-54, e.g., due to loss of exon 3; and “αS-112” alpha-synuclein-112, which lacks residue 103-130, e.g., due to loss of exon 5.

In one aspect, the monomeric αS substrate comprises, consists essentially of, or consists of wild type or recombinant human αS protein having 140 amino acids, having a molecular mass of 14,460 Da, and being represented by the sequence:

SEQ ID NO. 1:
MDVFMKGLSK AKEGVVAAAE KTKQGVAEAA GKTKEGVLYV
GSKTKEGVVH GVATVAEKTK EQVTNVGGAV VTGVTAVAQK
TVEGAGSIAA ATGFVKKDQL GKNEEGAPQE GILEDMPVDP
DNEAYEMPSE EGYQDYEPEA

In some aspects, the monomeric αS substrate comprises, consists essentially of, or consists of a conservative variant of SEQ ID NO. 1. A conservative variant may be a peptide or amino acid sequence that deviates from SEQ ID NO. 1 only in the substitution of one or several amino acids for amino acids having similar biochemical properties and having a minimal or beneficial impact on the activity of the resultant protein in the αS-SAA. A conservative variant must functionally perform substantially like the base component, i.e., SEQ ID NO. 1. For example, a conservative variant of SEQ ID NO. 1 will aggregate with misfolded αS aggregate and will form aggregates with substantially similar reaction kinetics under similar reaction conditions. The conservative variant may have for example, one, two, three, four, five, six, seven (5%), and up to 14 (10%) substitutions in the amino acid sequence.

In some aspects, the monomeric αS substrate comprises a recombinant αS protein comprising six additional histidine amino acids (i.e., a polyHis purification tag) on the C-terminus of SEQ ID NO. 1, resulting in a molecular mass of 15,283 Da and being represented by the sequence:

SEQ ID NO. 2:
MDVFMKGLSK AKEGVVAAAE KTKQGVAEAA GKTKEGVLYV
GSKTKEGVVH GVATVAEKTK EQVTNVGGAV VTGVTAVAQK
TVEGAGSIAA ATGFVKKDQL GKNEEGAPQE GILEDMPVDP
DNEAYEMPSE EGYQDYEPEA HHHHHH

In some aspects, the monomeric αS substrate may be any of the monomeric αS substrates disclosed in U.S. Pat. No. 11,079,396 and conservative variants thereof. In some aspects, the monomeric αS substrate or conservative variant thereof specifically excludes a monomeric αS substrate consisting of SEQ ID NO: 1.

In some aspects, the monomeric αS substrate may be expressed and prepared as described in Shahnawaz, M. et al. Development of a Biochemical Diagnosis of Parkinson's Disease by Detection of alpha-Synuclein Misfolded Aggregates in Cerebrospinal Fluid. JAMA Neurol 74, 163-172 (2017), which is incorporated by reference herein in its entirety.

In some aspects, the monomeric αS substrate may be expressed and prepared as described in U.S. Pat. No. 11,254,718 (available from Amprion, Inc., cat #52020).

In some aspects, the method may include providing the monomeric αS substrate in labeled form. A labeled monomeric αS substrate may be considered a conservative variant. The monomeric αS substrate in labeled form may include one or more of: a covalently incorporated radioactive amino acid, a covalently incorporated, isotopically labeled amino acid, a covalently incorporated fluorophore, and the like. Thus, detection of the misfolded αS aggregate may include detecting the monomeric αS substrate in labeled form as incorporated into the amplified portion of misfolded αS aggregate.

The pre-incubation mixture may include various concentrations of the monomeric αS substrate as a function of the total volume of the pre-incubation mixture prior to conducting an incubation cycle. In some aspects, the pre-incubation mixture may include the monomeric αS substrate in a concentration, or in a concentration range, of: between about 500 nM and about 500 μM; between about 1 μM and about 200 μM; between about 5 μM to about 100 μM; between about 10 μM and about 50 μM; between about 50 μM and about 75 μM; about 65 μM (i.e., about 1 mg/ml); 65 μM; between about 10 μM and about 30 μM; greater than 10 μM and less than 30 μM; about 20 μM; about 19.6 μM (i.e., about 0.3 mg/ml); or 19.6 μM. In one aspect, the pre-incubation mixture includes a concentration of the monomeric αS substrate as a function of the total volume of the pre-incubation mixture prior to conducting an incubation cycle of about 0.3 mg/ml.

### Buffer Compositions

The pre-incubation mixture may include various buffer compositions. The buffer composition may be effective to maintain the pH of the reaction mixture in a range from about pH 5 to about pH 9, from about pH 6 to about pH 8, from about pH 6 to about pH 7, from about pH 7 to about pH 8, about pH 7, about pH 7.4, from about pH 6.2 to about pH 6.5, including pH 6.3, 6.4, and 6.5. In one aspect, the buffer composition may be effective to maintain the pH of the reaction mixture at about 6.5. In some aspects, the pre-incubation mixture comprises one or more of the buffers Tris-HCL, IVIES, PIPES, MOPS, BES, TES, and HEPES. In some aspects, the buffer comprises PIPES in a concentration of about 100 mM, about 200 mM, about 300 mM, about 400 mM, about 500 mM, about 600 mM, or about 700 mM. In one aspect, the buffer comprises PIPES in a concentration of about 100 mM.

### Salt Solutions

In some aspects, the pre-incubation mixture comprises salt in a given concentration. The salt may, for example, enhance signal to noise ratio in fluorescence detection. In one aspect, the salt comprises NaCl. Other suitable salts may include KCl. In one aspect, the salt, e.g., NaCl, may be present in the pre-incubation mixture in a concentration of about 50 mM to about 1,000 mM, about 50 mM to about 500 mM, about 50 to about 150 mM, about 150 mM to about 500 mM, about 50 mM, about 150 mM, about 300 mM, about 500 mM, about 600 mM, or about 700 mM. In one aspect, the salt, e.g., NaCl, is present in a concentration of about 500 mM.

### Indicators

In some aspects, pre-incubation mixture comprises an indicator to determine if a detectable amount of misfolded αS aggregate is present in the reaction mixture. The indicator can be characterized by exhibiting an indicating state in the presence of a detectable amount of misfolded αS aggregate and a non-indicating state in the absence of a detectable amount of misfolded αS aggregate. Determining the presence of misfolded αS aggregate in a biological sample may include detecting the indicating state of the indicator of misfolded αS aggregate. The indicating state of the indicator and the non-indicating state of the indicator may be characterized by a difference in fluorescence. Thus, the step of determining the presence of misfolded αS aggregate in a biological sample may include detecting the difference in fluorescence. In some aspects, a molar excess of the indicator may be used, the molar excess being, for example, greater than a total molar amount of the monomeric αS substrate and the misfolded αS aggregate in the reaction mixture.

In some aspects, the indicator comprises a fluorophore. In some aspects, the indicator may include one or more of: Thioflavin-T (ThT), Congo Red, m-I-Stilbene, Chrysamine G, P113, BF-227, X-34, TZDM, FDDNP, IMPY, NIAD-4, luminescent conjugated polythiophenes, a fusion with a fluorescent protein such as green fluorescent protein and yellow fluorescent protein, derivatives thereof, and the like. A suitable indicator is ThT. In one aspect, wherein the indicator comprises ThT, the ThT concentration in the pre-incubation mixture is between about 5 μM and about 10 μM. In one aspect, wherein the indicator comprises ThT, the ThT concentration in the pre-incubation mixture is 5 μM.

### Sarkosyl

In some aspects, the pre-incubation mixture comprises sarkosyl. In some aspects, the sarkosyl is present in a concentration of 0.01% w/v to about 1.0% w/v. In some aspects, the sarkosyl is present in a concentration of 0.05% w/v to about 0.2% w/v. In some aspects, the sarkosyl is present in a concentration of about 0.1% w/v.

### Incubation Conditions

The reaction mixture may be held within a suitably sized container, such as a multi-well plate having a plurality of wells. For example, the multi-well plate may include 96 wells. The wells of the multi-well plate may have a volume of from 100 μL to 1000 μL, from 150 μL to 750 μL, or from 200 μL to 350 μL. In some aspects, at least one well of the multi-well plate contains one or more beads.

The temperature of the reaction mixture, in each incubation cycle, at a temperature in ° C., can independently be about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or a range between any two of the preceding values, for example, between about 15° C. and about 50° C., or between about 25° C. and about 45° C., or between about 30° C. and about 42° C. In some aspects, the incubation is carried out at about normal physiological temperatures for a warm-blooded animal. In further aspects, incubating the reaction mixture is conducted at a temperature between about 35° C. and about 45° C. or between about 37° C. and about 42° C. In one aspect, the method comprises incubating the reaction mixture at a temperature of about 42° C.

In several aspects, de-aggregating the incubation mixture may include subjecting the incubation mixture to physical disruption, such as shaking, sonication, stirring, freezing/thawing, laser irradiation, autoclave incubation, high pressure, homogenization, and the like. Shaking may include cyclic agitation, such as orbital agitation. The cyclic agitation may be conducted between about 50 rotations per minute (RPM) and 10,000 RPM. The cyclic agitation may be conducted between about 200 RPM and about 2000 RPM. The cyclic agitation may be conducted at about 500 RPM or about 600-800 RPM. In one aspect, the shaking comprises orbital agitation at about 800 RPM. De-aggregation of the incubation mixture may be conducted after each incubation cycle for between about 5 seconds and about 10 minutes, between about 30 seconds and about 1 minute, between about 45 seconds and about 1 minute, for about 1 minute, and the like.

The steps of incubating and de-aggregating the reaction mixture are repeated as necessary to amplify the misfolded αS aggregate of the biological sample to provide a detectable amount of misfolded α-S aggregate. Incubating the reaction mixture and de-aggregating the reaction mixture constitute an incubation cycle. The incubation cycle may be repeated between once and about 1000 times, between two times and about 500 times, between about 50 times and about 500 times, between about 150 times and about 250 times, and the like. In one aspect, for the final round of the incubation cycle, it may be advantageous to omit the de-aggregation step before performing the detecting step.

An incubation cycle may be carried out for a time between about 1 minute and about 5 hours, between about 10 minutes and about 2 hours, between about 15 minutes and about 1 hour, between about 25 minutes and about 45 minutes, and the like. In some aspects, incubating the reaction mixture and de-aggregating at least a portion of the misfolded αS aggregate includes an incubation cycle lasting from about 0.1 to 1 hours. Each incubation cycle may include independently incubating and de-aggregating the reaction mixture for one or more of: incubating between about 1 minute and about 5 hours and de-aggregating between about 5 seconds and about 10 minutes; incubating between about 10 minutes and about 2 hours and de-aggregating between about 30 sec and about 1 minute; incubating between about 14 minutes and about 1 hour and de-aggregating between about 45 seconds and about 1 minute; incubating between about 25 minutes and about 45 minutes and de-aggregating between about 45 seconds and about 1 minute; and incubating about 1 minute and de-aggregating about 1 minute. In one aspect, each incubation cycle includes incubating for about 14 minutes and de-aggregating for about 1 minute.

### Beads

In some aspects, the pre-incubation mixture may include one or more beads. Beads are small, typically spherical objects such as high-density beads. Including beads in the reaction mixture increases the rate of elongation of misfolded αS aggregates from the monomeric αS substrate and the soluble, misfolded αS protein of the biological sample. These beads are different in composition and function from the antibody coated magnetic or paramagnetic beads or particles (e.g., Dynabeads) used in concentration and/or immune depletion steps as described elsewhere herein.

The beads may be comprised of a variety of materials. For example, in some aspects, the beads are comprised of silicone, ceramic material, silica, glass, borosilicate glass, or Si₃N₄.

In some aspects, the beads comprise, consist essentially of, or consist of Si₃N₄. In some aspects, the beads comprise, consist essentially of, or consist of borosilicate glass. In one aspect, zirconium/silica beads are excluded. In one aspect, glass beads other than borosilicate glass beads are excluded.

In some aspects, the beads included in the incubation mixture may have a mean diameter of greater than 0.5 mm. In some aspects, the beads have a mean diameter from greater than 0.5 to about 10 mm. In some aspects, the beads have a mean diameter from greater than 0.5 mm to about 5 mm. In further aspects, the beads have a mean diameter ranging from greater than 0.5 mm to about 3.5 mm. In some aspects, the beads have a mean diameter from about 1.0 to about 10 mm, while in additional aspects the beads have a mean diameter from about 1.0 mm to about 5 mm. In further aspects, the beads have a mean diameter ranging from greater than 1.0 mm to about 3.5 mm. In some aspects, the beads have a mean diameter from 2.38 mm to about 10 mm, while in additional aspects the beads have a mean diameter from 2.38 mm to about 5 mm. In further aspects, the beads have a mean diameter ranging from greater than or equal to about 2.3 mm to about 3.5 mm, from about 2.38 to about 3.5 mm, or from about 2.45 mm to about 3.5 mm. In further aspects, the beads may have a mean diameter from about 1 mm to about 5 mm, from greater than 2.3 mm to about 5 mm, from greater than 3 mm to about 5 mm, about 2.38 mm, about 2.45 mm, or about 3.175 mm. In some aspects, the beads comprise, consist essentially of, or consist of Si₃N₄, have a mean diameter of 2.38 mm, and are blocked with bovine serum albumin (BSA). In some aspects, the beads comprise, consist essentially of, or consist of Si₃N₄, have a mean diameter of 3.175 mm, and are unblocked. In some aspects, the beads comprise, consist essentially of, or consist of borosilicate glass, have a mean diameter of 2.45 mm, and are unblocked. In some aspects, beads having a mean diameter of 2.3 mm or less are excluded from the invention. In some aspects, glass beads having a mean diameter of 2.3 mm or less are excluded from the invention. In some aspects, beads having a mean diameter of 3 mm or less are excluded from the invention. The size distribution of the beads is defined so that more than 90% of the beads are found between 80-120% of the mean bead diameter or between 90-110% of the mean bead diameter.

The number of beads included in the pre-incubation mixture can vary. In some aspects, the pre-incubation mixture consists of one bead. In some aspects, the pre-incubation mixture consists of two beads. In some aspects, the pre-incubation mixture comprises a plurality of beads. In one aspect, the pre-incubation mixture consists of two unblocked ⅛″ (3.175 mm) Si₃N₄ beads.

In some aspects, the surface of the one or more beads is “blocked” with a protein. Blocking the surface of the bead with a protein refers to providing a coating or layer over all or a substantial portion of the surface of the bead. Any suitable biocompatible protein can be used to coat the surface of the bead. A suitable protein for use in blocking the surface of the bead is an albumin, such as BSA. Other suitable blocking proteins may include casein or milk powder. The one or more beads can be blocked by soaking the one or more beads in a solution including the protein. The solution can be a water solution and/or a buffered solution such as PIPES, Tris-HCl, MES, MOPS, BES, TES, and HEPES.

As noted above, in some aspects, the incubation mixture is contained in a multi-well plate including a plurality of wells. For example, the multi-well plate can include 96 wells. In one aspect, such as when the beads are Si₃N₄ beads, the container may be a black bottom 96-well plate (Costar 3916). In one aspect, such as when the beads are Si₃N₄ beads, the container may be a bottom-read Greiner plate. In one aspect, such as when the beads are borosilicate glass beads, the container may be a clear bottom 96-well plate (Costar 3603). In one aspect, the container may be a Fisherbrand black bottom plate (cat #12-566-620).

### Detection

Detection includes repeating the steps of incubating and de-aggregating the reaction mixture as necessary to amplify sufficient misfolded αS aggregate present in the biological sample to provide an amplified incubation mixture having a detectable amount of misfolded αS aggregate. The incubation mixture may be contacted with an indicator, and the level of fluorescence of the amplified reaction mixture may be determined.

A suitable indicator is ThT, which is also known as Basic yellow 1. When ThT is added to samples containing β-sheet-rich deposits, such as the cross-β-sheet quaternary structure of amyloid fibrils, ThT fluoresces strongly with excitation and emission maxima at about 435 nm (or about 440 nm, depending on the fluorometer or spectrofluorometer) and about 485 nm (or about 490 nm, depending on the fluorometer or spectrofluorometer), respectively.

ThT fluorescence is typically measured by fluorescence spectroscopy using a filter fluorometer or spectrofluorometer. In some aspects, the ThT fluorescence emission intensity may be compared to the level of a corresponding control sample when carrying out the analysis to quantify the amount of misfolded αS aggregate in the biological sample.

Once the ThT fluorescence level has been determined, it can be displayed in a variety of ways. For example, the levels can be displayed graphically on a display as numeric values, proportional bars (i.e., a bar graph), or any other display method known to those skilled in the art.

An increase in the level of fluorescence indicates the presence of αS aggregate in the biological sample. In some aspects, a significant increase in the level of fluorescence indicates the presence of αS aggregate in the biological sample. In some aspects, a “significant increase” is an increase in the level of fluorescence of the incubated mixture at maximum fluorescence of at least two times the standard deviation of the fluorescence of the incubated mixture at maximum fluorescence compared to the level of fluorescence of the incubated mixture at any point during the lag phase indicates the presence of αS aggregate in the biological sample.

### Neurological Disorders and Synucleinopathies

αS aggregation may be associated with protein misfolding disorders (PMDs), e.g., PD, DLB, and MSA. However, existing technology is not clear whether this aggregation phenomenon is the cause of these diseases; it is suspected that these misfolded αS aggregates may cause cell dysfunction and tissue damage, among other effects.

In one aspect, a method is provided for diagnosing or aiding in the diagnosis of PD, DLB, MSA, or a spectrum of aspects of each in a subject having a neurological disorder. A neurological disorder is any disorder of the nervous system. Examples of neurological disorders include movement disorders such as PD, autonomic nervous system diseases such as MSA, and neuropsychiatric illnesses such as DLB.

In some aspects, the neurological disorder is a synucleinopathy. Synucleinopathies are neurodegenerative diseases characterized by the abnormal accumulation of aggregates of αS in cells of the nervous system such as neurons, nerve fibers, and glial cells. In some aspects, the synucleinopathy has symptoms associated with PD, DLB, or MSA, including, e.g., impaired cognition, sleep disorders, and gastrointestinal tract dysfunction.

In some aspects, the sample may be taken from a subject exhibiting no clinical signs of PD, DLB, or MSA. In other aspects, the biological sample may be taken from a subject exhibiting clinical signs of PD, MSA, DLB, or any combination thereof. The most recognizable symptom of PD is motor-related dysfunction.

In some aspects, the method includes treating a subject diagnosed as having PD with treatment for PD and/or its symptoms. Deep brain stimulation can be used to reduce motor symptoms associated with PD. Drugs useful for treating the motor symptoms of PD include levodopa, dopamine agonists, and monoamine oxidase B inhibitors. However, additional treatments for PD continue to be developed. See Radhakrishnan D M, Goyal V, Neurol India., 66(Supplement):S26-S35 (2018) and Iarkov et al., Front Aging Neurosci., 12:4 (2020).

### Supplemental Diagnostic Tests

In some aspects, the method may further comprise additional tests to confirm the αS-SAA-based indication, for example, to further distinguish the misfolded αS aggregates from a patient indicated by αS-SAA to have PD from the misfolded αS aggregates from a patient indicated by αS-SAA to have MSA or DLB. Examples of additional tests include the use of ligands having a high affinity for one of PD or MSA or DLB misfolded αS aggregates, creating a profile of protease-resistant fragments from the misfolded αS aggregate, and evaluating the structure of the detected misfolded αS aggregate using CD, FTIR, or cryo-ET.

### Kits

Another aspect provides a kit for detecting the presence of misfolded αS aggregate in a biological sample. The kit includes a known amount of a monomeric αS substrate; a known amount of an indicator; a buffer composition; optionally one or more beads having a mean diameter from about 1 mm to about 5 mm, from greater than 2.3 mm to about 5 mm, from greater than 3 mm to about 5 mm, about 2.38 mm, about 2.45 mm, or about 3.175 mm; optionally sarkosyl; and instructions directing a user to carry out the method of detecting of misfolded αS aggregate as described herein. The kit should also include a package for holding the components of the kit.

A kit generally includes a package with one or more containers holding the reagents, as one or more separate compositions or, optionally, as an admixture where the compatibility of the reagents will allow. The kits may further include buffers, labeling agents, controls, and any other materials necessary for carrying out the detection of misfolded αS aggregate. Kits can also include a tool for obtaining a sample from a subject, such as a swab or other biological fluid collection device.

The kit can also include instructions for using the kit to carry out a method of guiding treatment of a synucleinopathy in a subject. Instructions included in kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The kit may include one or more of: a bead distribution apparatus; a multi-well plate including a plurality of wells; a microfluidic plate; a shaking apparatus; an incubating apparatus; and a fluorescence measurement apparatus; included either as one or more of the individual plates or apparatuses, or as a combination device. For example, a shaking microplate reader may be used to perform cycles of incubation and shaking and automatically measure the ThT fluorescence emission during an experiment (e.g., FLUOstar OPTIMA, BMG LABTECH Inc., Cary, N.C. or Buehler Shaker TIMIX 5 shaker).

### EXAMPLES

The complete disclosure of all patents, patent applications, and publications, and electronically available material cited herein are incorporated by reference. The foregoing detailed description and the following examples have been given for clarity of understanding only. No unnecessary limitations are to be understood therefrom. The invention is not limited to the exact details shown and described, for variations obvious to one skilled in the art will be included within the invention defined by the claims.

### Example 1: Detection of Misfolded αS Aggregate in Human Blood Plasma by Immune-Capture Approach

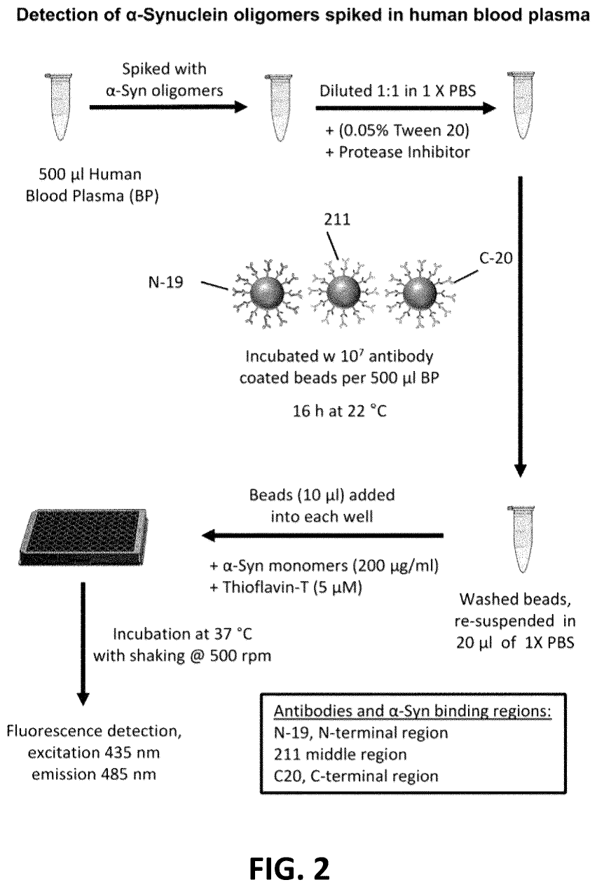

FIG. 2 is a schematic representation of an immunoprecipitation method for detecting the presence of αS aggregate in a blood plasma sample. First, anti-α-synuclein (211 and C-20) coated beads (1×10⁷ Dynabeads) were incubated with human blood plasma (500 μl) as such or spiked with different quantities of αS seeds (2000 to 0.2 pg). After immunoprecipitation, 10 μl of the immunoprecipitated Dynabeads were added to each well of a 96-well plate. Also added to the wells was a pre-reaction mixture comprising: (1) monomeric αS substrate (100 μg/ml) corresponding to SEQ ID NO: 2; (2) a buffer composition comprising 100 mM PIPES, pH=6.5; (3) a salt solution comprising 500 mM NaCl; (4) an indicator comprising 504 ThT; and (5) 0.05% Tween-20, to form a reaction mixture. The reaction mixture was incubated at 37° C. with intermittent agitation cycles (orbital, 500 RPM; 1 min shaking: 29 min incubating) to form an incubated reaction mixture. The incubated reaction mixture was illuminated (435 nm excitation; 485 nm emission), and the increase in the level of fluorescence was monitored.

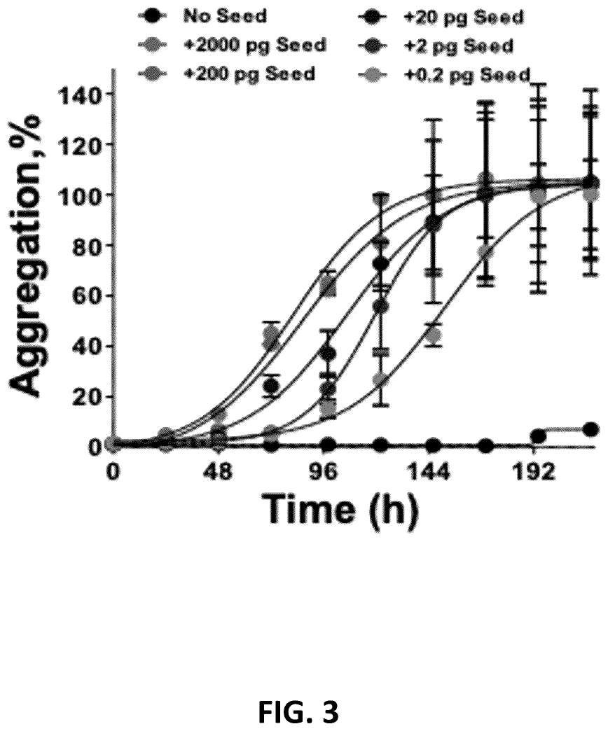

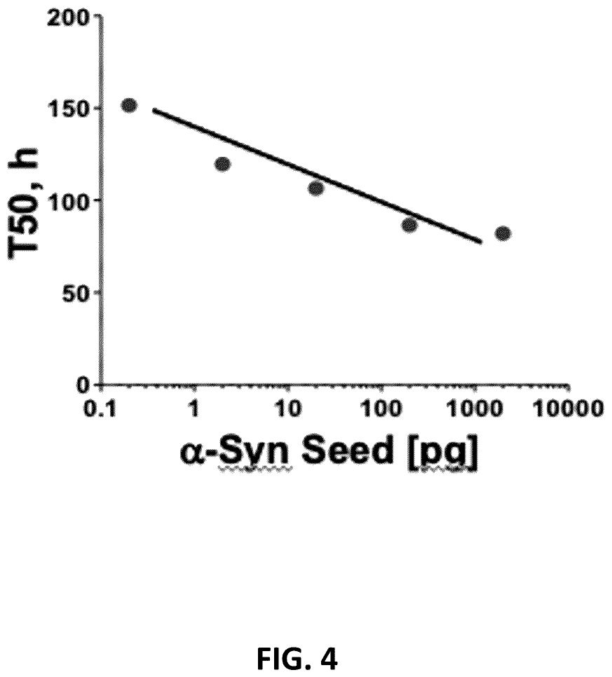

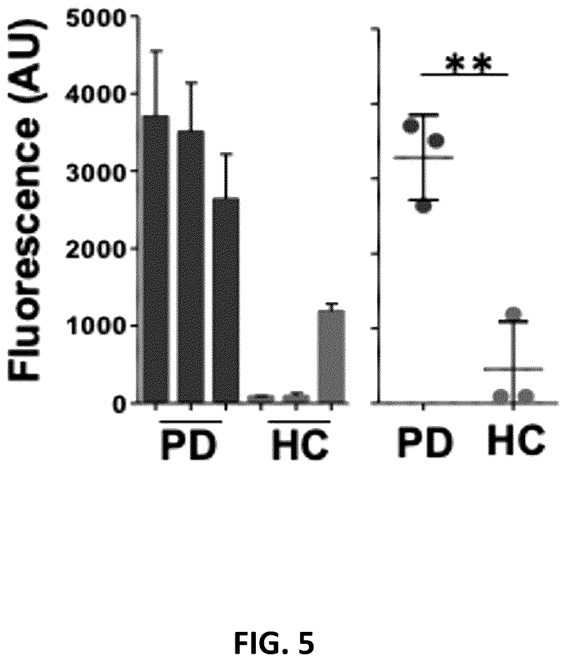

FIG. 3 shows αS-SAA aggregation curves using the various concentrations of αS synthetic seeds. FIG. 4 is a plot of time to reach 50% aggregation (T50) as a function of the logarithm of αS synthetic seed concentration. FIG. 5 is a graph showing maximum ThT fluorescence (left) during αS-SAA using human blood plasma from patients diagnosed as having PD compared to HCs. Statistical significance was evaluated by t-test (right).

### Example 2: Detection of Misfolded αS Aggregate in Human Blood Plasma by Sedimentation Approach

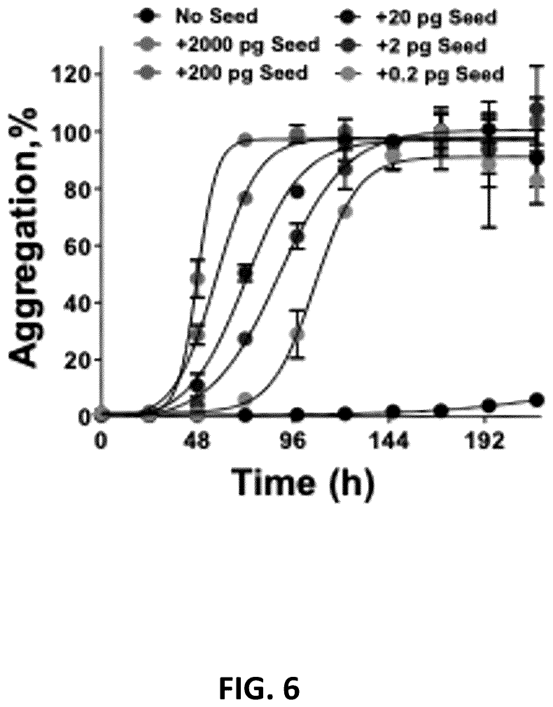

Either different concentrations of synthetic αS seeds were added into healthy plasma or blood plasma from a PD patient was used. Samples were centrifuged at 100,000×g for 1 h, and the pellet was directly resuspended in a pre-incubation mixture, the pre-incubation mixture comprising: (1) 1 mg/ml of a monomeric αS substrate corresponding to SEQ ID NO: 2; (2) a buffer composition comprising 100 mM PIPES, pH=6.5; (3) a salt solution comprising 500 mM NaCl; (4) an indicator comprising 5 μM ThT; and (5) 1% sarkosyl, to form a reaction mixture. The reaction mixture was incubated at 37° C. with intermittent agitation cycles (orbital, 500 RPM; 1 min shaking: 29 min incubating) to form an incubated reaction mixture. The incubated reaction mixture was illuminated (435 nm excitation; 485 nm emission), and the increase in the level of fluorescence was monitored. The aggregations curves corresponding to the varying synthetic αS seed quantities are shown in FIG. 6.

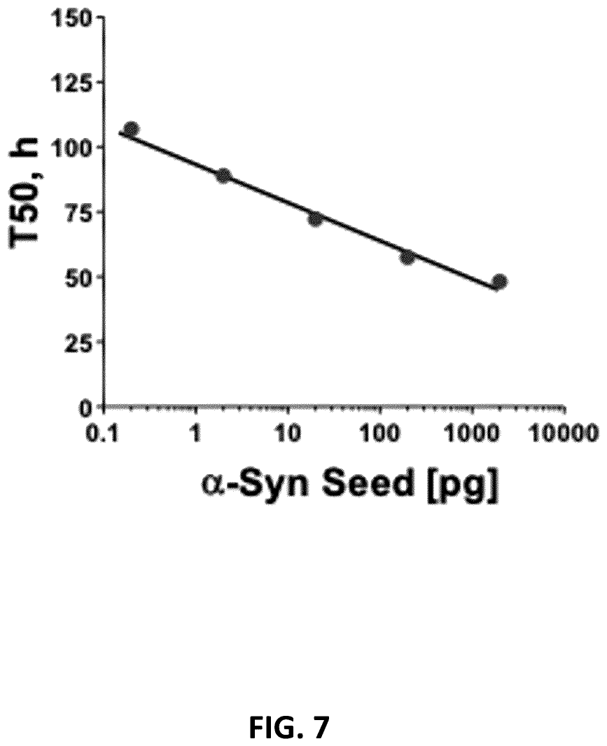

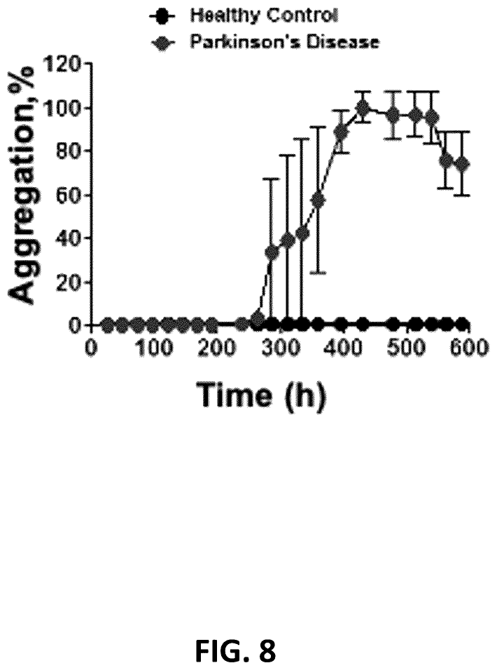

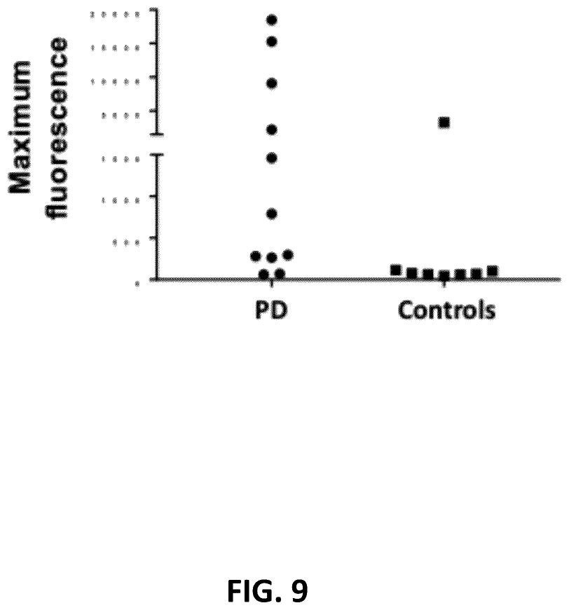

To illustrate the direct relationship between the quantity of aggregates present in spiked blood samples and the kinetic parameters of the αS-SAA, the T50 obtained in FIG. 6 was plotted against the logarithmic mass of αS seeds. The result is shown in FIG. 7. FIG. 8 shows a representative aggregation profile of monomeric αS substrate in the presence of concentrated blood plasma from a HC and PD patient. Finally, FIG. 9 shows a summary of the results obtained with 11 PD and eight HC biological samples, displaying the maximum fluorescence intensity.

### Example 3: Detection of Misfolded αS Aggregate in Human Blood Plasma by Sedimentation Approach

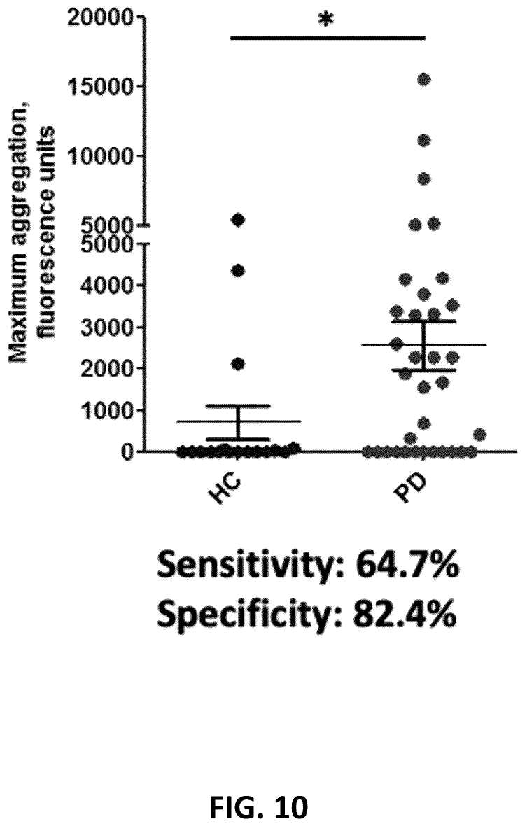

Human blood plasma samples (500 μl) from healthy controls (HC; n=17) and PD patients (PD; n=34) were processed according to the procedure described in Example 2, except that the pellets were washed 3 times with 1×PBS and re-suspended in 1×PBS prior to being added to the well plate. FIG. 10 shows the maximum ThT fluorescence. Significance was evaluated by t-test.

### Example 4: Detection of Misfolded αS Aggregate in Skin Homogenates

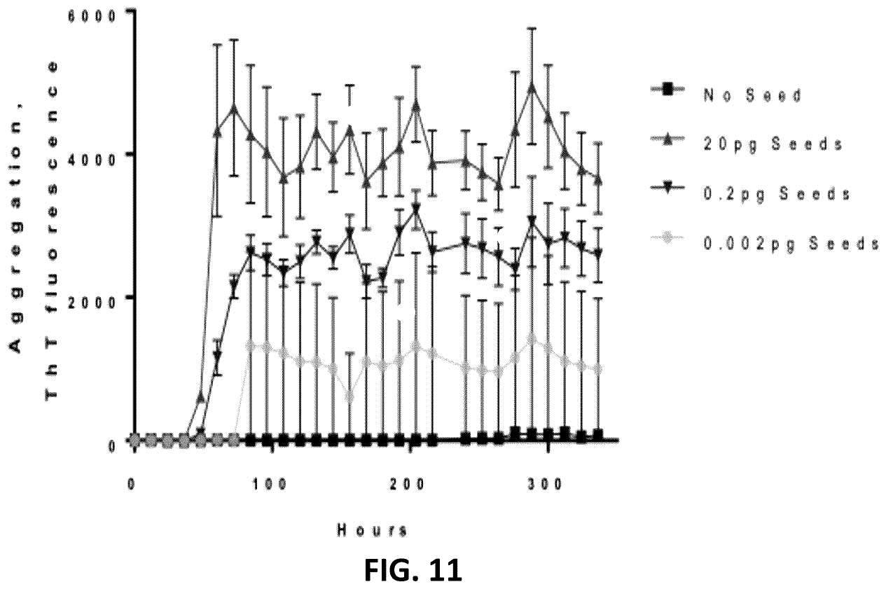

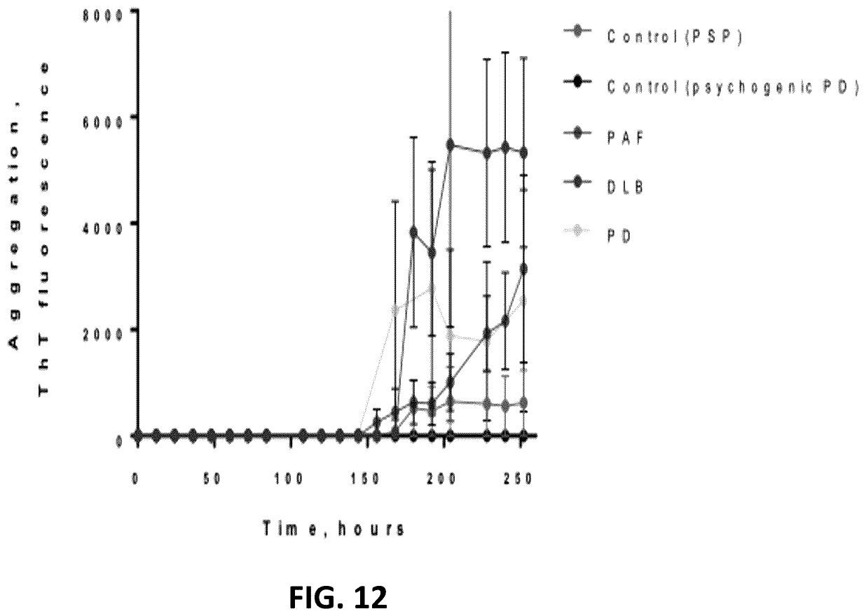

Either a 0.01% skin homogenate was spiked with different concentrations of synthetic αS seeds (0 (No seeds), 20, 0.2, and 0.002 pg) or 0.01% skin homogenates from patients diagnosed as having one of various synucleinopathies were used. The spiked skin homogenates were combined with a pre-incubation mixture, the pre-incubation mixture comprising: (1) 1 mg/ml of a monomeric αS substrate corresponding to SEQ ID NO: 2; (2) a buffer composition comprising 100 mM PIPES, pH=6.5; (3) a salt solution comprising 500 mM NaCl; (4) an indicator comprising 5 μM ThT, to form a reaction mixture. The reaction mixture was incubated at 37° C. with intermittent agitation cycles (orbital, 500 RPM; 1 min shaking: 29 min incubating) to form an incubated reaction mixture. The incubated reaction mixture was illuminated (435 nm excitation; 485 nm emission), and the increase in the level of fluorescence was monitored. The aggregations curves corresponding to the varying synthetic αS seed concentration are shown in FIG. 11. FIG. 12 shows the results using skin homogenates from patients diagnosed as having PD, DLB, or PAF, as well as negative controls, including psychogenic PD or other neurodegenerative diseases (OND) such as progressive supranuclear palsy (PSP). The study was performed in triplicate, and the data show mean±SEM.

### Example 4: Detection of Misfolded αS Aggregate in Skin Homogenates

4.1 Preprocessing. Homogenization Protocol.

1×PBS was prepared and supplemented with Complete Protease Inhibitor Cocktail EDTA-free (1×PBS-PI). A skin sample was transferred to a pre-weighed homogenization tube containing 6 stainless steel beads and weighed. An amount of 1×PBS-PI necessary to produce a 10% or a 1% w/v skin homogenate was added to the skin sample in the homogenization tube. The skin sample/1×PBS-PI mixture was placed in a homogenizer and homogenized according to the manufacturer's instructions, until the skin sample was visibly fully homogenized. The skin homogenate prepared in 1×PBS-PI was transferred to a centrifugation tube and centrifuged at 800×g for 1 min at 4° C. (condition A); 1,000×g for 10 min at 4° C. (condition B); or 10,000×g for 10 min at 4° C. (condition C). The supernatant was collected, vortexed, aliquoted, snap-frozen, and stored at −80° C. until use.

### 4.2 Preprocessing. αS-SAA Sample Preparation.

The skin homogenate was thawed and tested undiluted or diluted 10⁻⁴ and 10⁻⁵ in synthetic CSF (available from Amprion, Inc., cat #52022) (the composition of synthetic CSF or “sCSF” is described in U.S. Publication No. US20230084155A1, the entire disclosure of which is incorporated herein by reference).

### 4.3 αS-SAA.

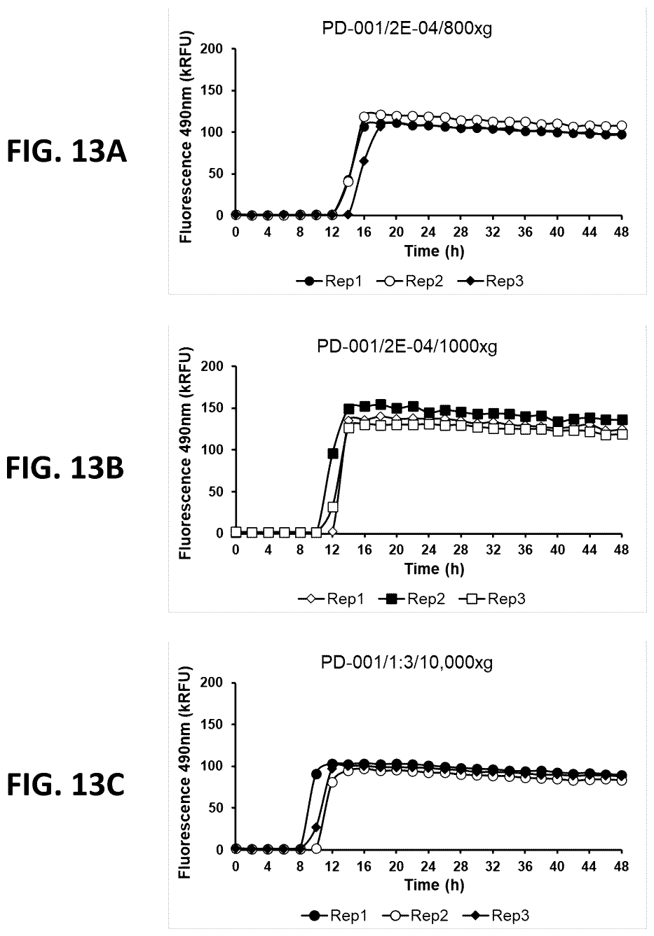

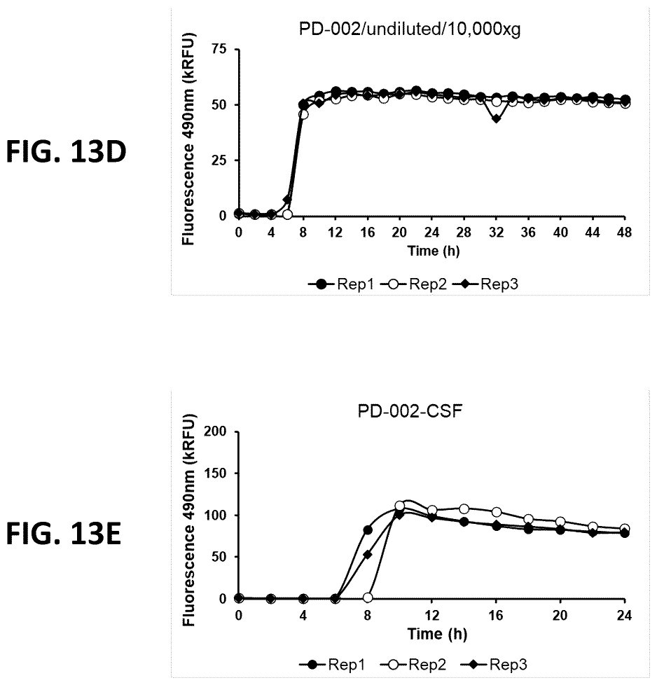

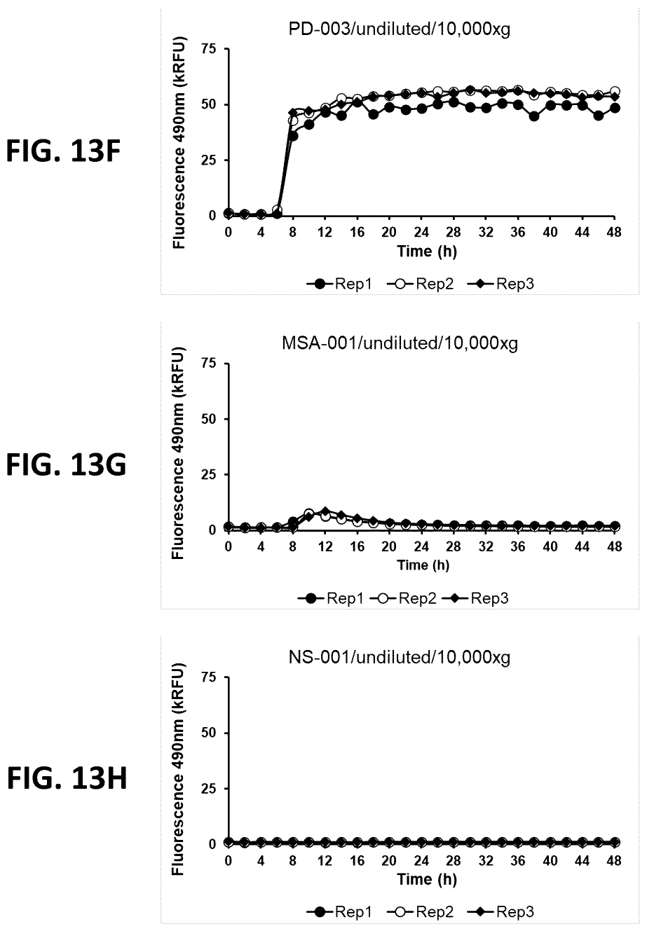

40 μL of undiluted or diluted skin homogenate was combined with 60 μL of a pre-incubation mixture in a bottom-read Greiner clear bottom plate or a Fisherbrand black bottom plate, the pre-incubation mixture comprising: (1) a monomeric αS substrate (0.3 mg/ml) corresponding to SEQ ID NO: 2; (2) a buffer composition comprising 100 mM PIPES, pH=6.5; (3) a salt solution comprising 500 mM NaCl; (4) an indicator comprising 5 μM ThT; (5) 0.1% sarkosyl; and (6) two unblocked ⅛″ (3.175 mm) grade 5 Si₃N₄ beads, to form a reaction mixture. The reaction mixture was incubated (orbital, 600-800 RPM, 1 min shaking: 14 min incubating, 42° C., for a total 15 min cycle) for 24 h in a BMG reader/shaker or Buehler Shaker TIMIX 5 shaker to form an incubated reaction mixture. The incubated reaction mixture was intermittently illuminated (about 440 nm excitation; about 490 nm emission), and the increase in the level of fluorescence was monitored. FIGS. 13A-13C show αS-SAA aggregation curves under centrifugation conditions A-C, respectively, from diluted skin homogenate from a patient diagnosed as having PD. FIGS. 13D and 13E show the αS-SAA aggregation curves from the skin and CSF, respectively, of the same patient diagnosed with PD. FIGS. 13F and 13G show αS-SAA aggregation curves under centrifugation condition C from skin homogenates from patients diagnosed as having PD and MSA, respectively. FIG. 1311 shows S-SAA aggregation curves under centrifugation condition C from skin homogenates from a non-synucleinopathy (NS) control subject.

### Example 5: Detection of Misfolded αS Aggregate in OM

### 5.1 Collection and Pre-Processing.

The patients were subjected to local anesthesia (nasal spray with lidocaine) 10 min before the procedure. Through a rigid fiberscope, the OM (between septum and middle turbinate) is identified. Keeping the fiberscope in place, a cotton swab was inserted into the nostril, and once it reached the OM, the wall of the nostril was gently scratched to collect the sample. The swab was removed from the nose and placed in a 15 mL conical tube containing 3 mL of physiological solution (saline buffer). Disposable scissors were used to cut the steam of the swab so that it fit inside the 15 mL conical tube. The tube was vortexed for 1 min. Using disposable tweezers, the swab was transferred to a second 15 mL conical tube with 3 mL of physiological solution (saline buffer) and vortexed for 1 min. With the same disposable tweezers, the swab was transferred to a third 15 mL conical tube with 3 mL of physiological solution and vortexed for 1 min. The swab was discarded. 3 mL from each of the 15 mL tubes (9 mL total) was pooled into a single 15 mL tube, which was centrifuged 800×g for 20 min at 4° C. 8 mL of the supernatant saline solution was discarded. The pellet and 1 mL of saline were stored at −80° C.

### 5.2 Preprocessing. αS-SAA Sample Preparation.

An OM sample was collected from the pellet using a bacterial inoculation loop that holds ˜2 μg of sample. Three loops were collected (6 μg) and resuspended in 50 μL of 1×PBS (Sigma, cat #P5493-1L) by extensive vortex and pipetting up-and-down. The final resuspension is aliquoted in three single use aliquots containing 16.7 μL each (2 μg OM sample per aliquot). The sample was snap-frozen and stored at −80° C. until use.

### 5.3 Sample Processing for αS-SAA.

The OM/PBS sample was thawed and 4 μL of sample was pipetted into 76 μL of sCSF (Amprion, cat #S2022) to make a 1:20 dilution. A 1:400 (12 ng/40 μL) dilution was prepared by pipetting 24 μL of the 1:20 dilution into 456 μL of sCSF.

### 5.4 αS-SAA.

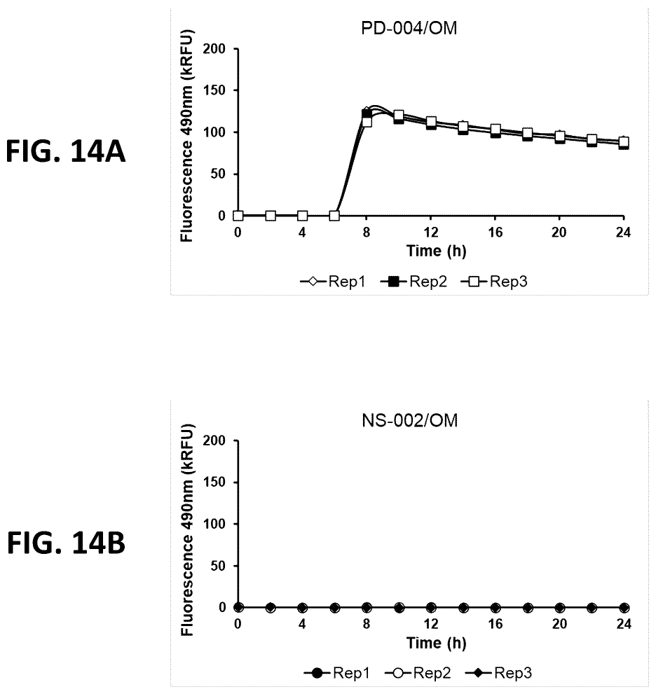

The reaction mixture contained 40 μL OM sample (12 ng and/or 24 ng) and 60 μL of pre-incubation mixture. The pre-incubation mixture included 100 mM PIPES pH 6.5, 0.3 mg/mL recombinant αS, 500 mM NaCl, 50 μM ThT, 0.1% sarkosyl, and two Si₃N₄ beads (⅛″, grade 5). Plates are orbitally shaken for 1 min at 600-800 RPM, followed by 14 min incubation, for a total 15 min cycle at 42° C., for 24 h. Fluorescence readings were taken at 440-10 nm (excitation) and 490-10 nm (emission). FIGS. 14A and 14B show αS-SAA aggregation curves from a patient diagnosed as having PD compared to a non-synucleinopathy control.

### Example 6: Detection of Misfolded αS Aggregate in Saliva (Prophetic)

### 6.1 Collection and Preprocessing.

Participants are required to avoid eating for at least 2 hours, smoking for at least 4 hours, and drinking alcohol for at least 12 hours before saliva collection. Each participant will open one clean 50-mL Falcon tube and collect saliva by drooling. Once completed, the Falcon tubes are immediately closed and placed on ice (raw saliva). 144 μL of saliva is taken from each sample and placed in a centrifugation tube. 6 μL of 25 mM PMSF-ethanol is added to 144 μL saliva for a 1 mM final concentration of PMSF and vortexed gently/briefly to mix well. The samples are centrifuged for 20 min at 12,000×g at 4° C. The supernatant from each sample is removed, and the processed saliva is transferred into a new 0.5-mL Eppendorf tube, snap-frozen, and stored at −80° C. until use.

### 6.2 αS-SAA.

The reaction contains 10 μL of processed saliva sample and 90 μL of pre-incubation mixture. The pre-incubation mixture includes 100 mM PIPES pH 6.5, 500 mM NaCl, 0.3 mg/mL recombinant αS, 10 μM ThT, 0.1% sarkosyl, and two silicone nitride beads (⅛″, grade 5). Plates are orbitally shaken for 1 min at 600-800 RPM, followed by 14 min incubation, for a total 15 min cycle at 42° C. Fluorescence readings are taken at 440-10 nm (excitation) and 490-10 nm (emission).

### Example 7: Detection of Misfolded αS Aggregate in Saliva Spiked with PD-CSF

### 7.1 Preprocessing.

Saliva samples from healthy control donors (HC), collected as specified above, were spiked with PD-CSF (2 parts of PD-CSF+8 parts of raw saliva). Spiked samples were incubated at room temperature for 1 min. PMSF-Ethanol was added to the spiked sample at a 1 mM final concentration. Samples were centrifuged at 12,000×g and 4° C. for 20 min. Supernatant from each sample were transferred into a new 0.5-mL Eppendorf tube and vortexed. Samples were snap-frozen and stored at −80° C. until assay.

### 7.2 αS-SAA

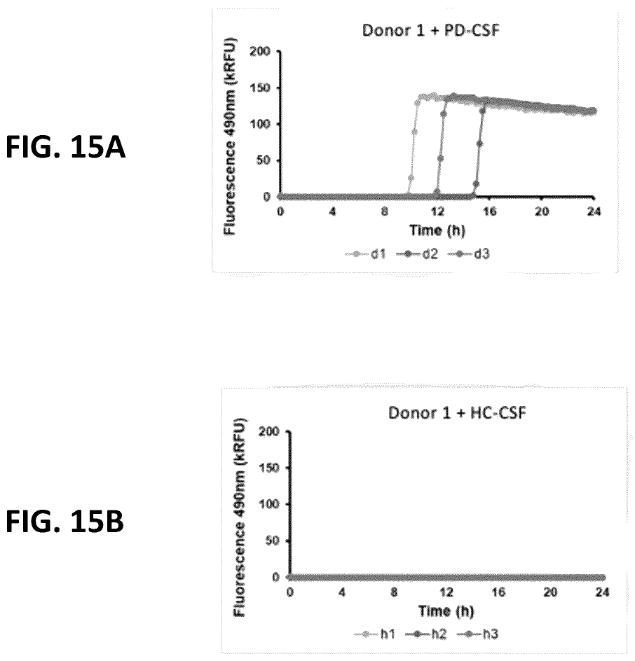

The reaction contains 10 μL of processed saliva sample and 90 μL of pre-incubation mixture. The pre-incubation mixture includes 100 mM PIPES pH 6.5, 500 mM NaCl, 10 μM ThT, 0.3 mg/mL recombinant αS, 0.1% sarkosyl, and two silicone nitride beads (⅛″, grade 5). Plates are orbitally shaken for 1 min followed by 14 min incubation, for a total 15 min cycle at 42° C., for 24 h. When using a robotic arm associated to the Omega shaker/reader (8 plates at the time), the agitation is set to 600 RPM, while stand-alone Omegas (1 plate at the time) are set to 800 RPM. Fluorescence readings were taken at 440-10 nm (excitation) and 490-10 nm (emission). FIGS. 15A-15B show actual αS-SAA aggregation curves of a HC saliva sample spiked with CSF from a patient diagnosed as having PD compared to a HC saliva sample spiked with HC CSF.

What is claimed is:

1. An in vitro method for detecting the presence of alpha-synuclein (αS) aggregate in a skin sample, the method comprising:
(A) providing a skin sample;
(B) providing a pre-incubation mixture, the pre-incubation mixture comprising:
(1) a monomeric αS substrate;
(2) a buffer composition;
(3) a salt solution;
(4) an indicator comprising a fluorophore;
(5) sarkosyl; and
(6) a bead,
(C) combining the skin sample and the pre-incubation mixture to form a reaction mixture;
(D) incubating the reaction mixture with an intermittent agitation cycle to form an incubated reaction mixture;
(E) illuminating the incubated reaction mixture with a wavelength of light that excites the fluorophore; and
(F) determining a level of fluorescence during incubation, wherein an increase in the level of fluorescence indicates the presence of αS aggregate in the skin sample.

2. The method of claim 1, wherein the monomeric αS substrate comprises SEQ ID NO: 2.

3. The method of claim 1, wherein the monomeric αS substrate comprises SEQ ID NO: 2 and is present in a concentration of 0.3 mg/ml±10%.

4. The method of claim 1, wherein the buffer composition comprises PIPES.

5. The method of claim 1, wherein the buffer composition comprises about 100 mM PIPES having a pH=6.5±10%.

6. The method of claim 1, wherein the salt solution comprises NaCl.

7. The method of claim 1, wherein the salt solution comprises about 500 mM NaCl.

8. The method of claim 1, wherein the indicator comprising a fluorophore comprises thioflavin T (ThT).

9. The method of claim 1, wherein the indicator comprising a fluorophore comprises thioflavin T (ThT) having a concentration of about 5 to about 10 μM.

10. The method of claim 1, wherein the sarkosyl is present in a concentration of about 0.1% w/v.

11. The method of claim 1, wherein the bead comprises silicone.

12. The method of claim 1, wherein the bead is a ceramic bead.

13. The method of claim 1, wherein the bead comprises glass.

14. The method of claim 1, wherein the bead comprises Si₃N₄.

15. The method of claim 1, wherein the bead comprises two Si₃N₄ beads, each having a diameter of 3.175 mm±10%.

16. The method of claim 1, wherein the increase in the level of fluorescence comprises an increase of greater than two times a standard deviation of a maximum fluorescence compared to the fluorescence at any point during a lag phase.

17. An in vitro method for detecting the presence of αS aggregate in an olfactory mucosa sample, the method comprising:
(A) providing an olfactory mucosa sample;
(B) providing a pre-incubation mixture, the pre-incubation mixture comprising:
(1) a monomeric αS substrate;
(2) a buffer composition;
(3) a salt solution;
(4) an indicator comprising a fluorophore;
(5) sarkosyl; and
(6) a bead,
(C) combining the diluted olfactory mucosa homogenate and the pre-incubation mixture to form a reaction mixture;
(D) incubating the reaction mixture with at least one agitation cycle to form an incubated reaction mixture;
(E) illuminating the incubated reaction mixture with a wavelength of light that excites the fluorophore; and
(F) determining a level of fluorescence during incubation, wherein an increase in the level of fluorescence indicates the presence of αS aggregate in the olfactory mucosa sample.

18. The method of claim 17, wherein the monomeric αS substrate comprises SEQ ID NO: 2.

19. The method of claim 17, wherein the monomeric αS substrate comprises SEQ ID NO: 2 and is present in a concentration of 0.3 mg/ml±10%.

20. The method of claim 17, wherein the buffer composition comprises PIPES.

21. The method of claim 17, wherein the buffer composition comprises about 100 mM PIPES having a pH=6.5±10%.

22. The method of claim 17, wherein the salt solution comprises NaCl.

23. The method of claim 17, wherein the salt solution comprises about 500 mM NaCl.

24. The method of claim 17, wherein the indicator comprising a fluorophore comprises thioflavin T (ThT).

25. The method of claim 17, wherein the indicator comprising a fluorophore comprises thioflavin T (ThT) having a concentration of about 5 to about 10 μM.

26. The method of claim 17, wherein the sarkosyl is present in a concentration of about 0.1% w/v.

27. The method of claim 17, wherein the bead comprises silicone.

28. The method of claim 17, wherein the bead is a ceramic bead.

29. The method of claim 17, wherein the bead comprises glass.

30. The method of claim 17, wherein the bead comprises Si₃N₄.

31. The method of claim 17, wherein the bead comprises two Si₃N₄ beads, each having a diameter of 3.175 mm±10%.

32. The method of claim 17, wherein the increase in the level of fluorescence comprises an increase of greater than two times a standard deviation of a maximum fluorescence compared to the fluorescence at any point during a lag phase.
